(19) United States
(12) Patent Application Publication
(10) Pub. No.: US 2018/0011973 A1
(43) Pub. Date: Jan. 11, 2018

(54) AN INTEGRATED MOBILE PERSONAL ELECTRONIC DEVICE AND A SYSTEM TO SECURELY STORE, MEASURE AND MANAGE USERS HEALTH DATA

(71) Applicant: OS - NEW HORIZONS PERSONAL COMPUTING SOLUTIONS LTD., Lod (IL)

(72) Inventors: Gila FISH, Mevasseret Zion (IL); Avner KORMAN, Herzlia (IL)

(21) Appl. No.: 15/547,077

(22) PCT Filed: Jan. 28, 2016
(86) PCT No.: PCT/IB2016/050432

§ 371 Date: Jul. 28, 2017

Related U.S. Application Data
(60) Provisional application No. 62/108,614, filed on Jan. 28, 2015.

(51) Int. Cl. G06F 19/00; G06F 21/62; H04L 29/06; A61B 5/00; A61B 5/0205; H04W 12/06; A61B 5/024; A61B 5/053; A61B 5/0476; A61B 5/0402; A61B 5/145; A61B 5/16; A61B 5/021

(52) U.S. Cl. CPC G06F 19/322; H04W 12/06; A61B 5/1171; G06F 21/6218; H04L 63/0861; A61B 5/0022; A61B 5/02055; A61B 5/1176; A61B 5/021; A61B 5/0533; A61B 5/0476; A61B 5/0402; A61B 5/4266; A61B 5/14532; A61B 5/14542; A61B 5/165; A61B 5/024

(57) ABSTRACT

A mobile device, methods and systems provide the invention mobile Personal Health Records (PHR) management platform solution. The platform enables secure PHR data management, measuring user's medical parameters, managing PHR secured depository containing user's health data on the user's invention combined phone & add-on sleeve device, while blocking none legitimate users access to the invention devices secured storage content. The invention device user's authentication is based on the combined weighted fusion of at least two different human biological sensors within the device and their weighted output analysis. The multi-sensors ensure bio-authentication secured memory entry only for the legitimate device user. In case of authentication success it activates various types of applications on the user PHR data depository content stored in device. The system supports the user's PHR remote health management, remotely monitoring the user's measured medical parameters, updating & managing user's health medical history depository in the user's electronic sleeve.

### FIELD OF THE INVENTION

The present invention relates to health data processing and secured health data management capabilities. For better sensitive health data access security management, there is a need for personal and highly secure legitimate device user's authentication, followed by the measurement and recording of user's personal health related parameters and further accumulating also the user's health related additional data received from external resources. The device should be functioning also as a vast content and secured health data depository. The invention should relate in particular to the integration of conventional smart mobile communication devices with computerized personal data measurement and storage devices to create a new type of mobile devices and their combined operational method. The legitimate device is user's authentication would enable the device exclusive use only by its owner, while blocking the device use and internal data depository content access to non-legitimate device users.

The Invention relates in particular also to the needs of supporting a specific health management application defined as the user's digital mobile Personal Health Records (mPHR), functioning as a personal mobile digital medical data depository, enabling the user's PHR creation and for further consistently updating and managing the owner's personal large scale and growing health history data depository. In addition the field of the invention deals with highly secured mobile, health related multi-users data management, storage and updating system, supporting the system's multi users with continuous personalized PHR data updating and management capabilities, done through cellular and internet communication. The system should also support measuring and sending out from the user's device a plurality of the user's recently measured medical data parameters, associated with the managing and supporting through the device its owner's health.

### BACKGROUND OF THE INVENTION

A personal health record, or PHR, is a health record where health data and information related to the care of a patient is maintained by the patient. This stands in contrast to the more widely used electronic medical record which is operated by institutions (such as hospitals) and contains data entered by clinicians or billing data to support insurance claims. The intention of a PHR is to provide a complete and accurate summary of an individual's medical history which is accessible online. The health data on PHR might include patient-reported outcome data, lab results, and data from devices such as wireless electronic weighing scales or collected passively from a smartphone.

The term “personal health record” is not new. The earliest mention of the term was in an article indexed by PubMed dated June 1978 and even earlier in 1956 reference is made to a personal health log. However, most scientific articles written about PHRs have been published since 2000. The term “PHR” has been applied to both paper-based and computerized systems; current usage usually implies an electronic application used to collect and store health data. In recent years, several formal definitions of the term have been proposed by various organizations.

based and computerized systems; current usage usually implies to an electronic application used to collect and store medical and health related data. In recent years several formal definitions of the term have been proposed by various organizations. It is important to note that PHRs are not the same as Electronic Health Records (EHRs). The latter are software systems designed for use by health care providers. Like the data recorded in paper-based medical records, the data in EHRs are legally mandated notes on the care provided by clinicians to patients. There is no legal mandate that compels a consumer or patient to store her personal health information in a PHR.

PHRs can contain a diverse range of data, including but not limited to: allergies and adverse drug reactions, chronic diseases, family history, illnesses and hospitalizations, imaging reports (e.g. X-ray, MRI, CT), laboratory test results, medications and dosing, prescription record, surgeries and other procedures, vaccinations, Etc.

There are two methods which data can arrive in a PHR. A patient may enter it directly, either by typing into fields or uploading/transmitting data from a file or another website. The second is when the PHR is tethered to an electronic health record, which automatically updates the PHR. Not all PHRs have the same capabilities, and individual PHRs may support one or all of these methods.

Portable wireless communication equipment, including smart mobile telephones, portable data assistants (PDAs), Notepads, Notebooks and other mobile electronic devices have been available and their technical and operational capabilities are fast expanding in the recent years. They are frequently used for implementing various daily needs through dedicated applications by the computer age users.

It has also been recognized that the high portability, strong computing power and the fast expanding daily frequent use of modern portable wireless communication equipment, is not any more just serving as a conventional phone or data communication device, but their use is expanding for using as modern communication devices as well as computerized mobile workstations. These computerized features are now serving most of modern user's life management needs. More specifically, because such devices are carried by most modern users through most of daily and night time activities hours, they are most suitable to be used as the user's safe and secured personal data depository including the user's personal and sensitive medical data depository. Further, a loss or theft of such devices creates a risk of unauthorized access and possible mal use of the device's internal user's personal data depository, the loss of a costly smartphone personal device containing a large variety of personal information important to its user, may lead to unauthorized access and mal-use by a hostile intruder to sensitive user's data stored on the device.

To reduce the risks of unauthorized use of the device's communication services and/or unauthorized access to stored data, most portable communication devices includes a password protection system. A typical password protection system is implemented by disabling the keypad, or the telephone circuits, and/or the specific installed data application, unless and until the user enters an applicable unlock code. Generally the password/unlock code is in the form of alpha numeric text which may be entered using the keypad of the mobile electronic device. There exist several challenges with such alphanumeric password/unlock code protection systems.

First, the protection provided by a password only exists so long as the password is not compromised. Many people tend to use passwords that are easily guessed, or write their passwords on paper, and otherwise compromise the integrity of their passwords.

Second, user entry of a password (and the associated key strokes needed to reach the password entry prompt and active the electronic device after password entry) can be hard to recall and also time consuming and aggravating—to the point where many people select the option of disabling the password protection of the mobile electronic device and their content.

An alternative system used to password protect a mobile telephone is disclosed in U.S. Pat. No. 6,351,634 to Shin. The system of Shin is useful for a mobile telephone that includes a touch screen. A registered secret symbol is used as the password. The secret symbol comprises a stroke number value responsive to the existence of pressure applied to the touch screen and X/Y coordinate values for each stroke. In operation, a user inputs a symbol using the pressure sensitive touch screen to draw the various strokes of the secret symbol. The device determines whether the input symbol matches the registered secret symbol and unlocks the telephone if the character stroke number value and the X/Y coordinate value signals match that of the secret password symbol. Shin teaches that the secret password symbol can be a character, a signature, a numeral, or a combination thereof. A significant challenge of the system of Shin is that so long as someone can duplicate the secret password symbol, whether by tracing the user's code insertion acts or by careful drawing possible variations on the touch screen, such person has access to the mobile telephone. Stated another way, authentication of the user is based on the user being able to duplicate the strokes and shape of the secret password symbol.

In a completely separate field of technology, character recognition has been proposed for use as a means for user input of character data into a computer system. For example, U.S. Pat. No. 6,188,392 to O'Connor discloses an electronic pen device that is coupled to a computer system by an RF transmitter or a batch communication docking station. The electronic pen device includes a combination of a pressure sensitive tip (for detecting contact with a surface) and accelerometers for detecting movement of the electronic pen device while in contact with the surface. Data from the accelerometers and the pressure sensitive tip are used to recognize each of a sequence of characters input by the user. While it may be possible to use the electronic pen device of O'Connor with a mobile telephone, such a system would include several drawbacks. First, such a system would be relatively expensive compared to a traditional mobile telephone or even the mobile telephone of Shin. Such a system would require the need for separate processing systems for both the mobile telephone and the pen, separate batteries, complimentary communication systems (whether by RF or docking station), and other duplicate components. Secondly, use of a discrete external electronic pen with a mobile telephone would be cumbersome at best for a user. The user would need to handle and maintain two separate devices.

The aim of a biometric system or module is the realization of the identification/authentication of people using some biological characteristic or physically measured behavior of the individual, in a safe and non-invasive way. The problem of identification and authentication of people is very old and has always tried in the past to be solved with different media: seals, titles, stamps, nameplates, etc. Today this is not enough and you need to introduce new legitimate user authentication and identification techniques to ensure that a person is who they say they are in many contexts.

There are many biometric techniques that try to recognize a person by their physical characteristics (iris, face morphology, fingerprint, voice recognition, etc.) or their behavior (gait, air gesture, manner of writing, online signature, etc.). It is vital in this document, by its similarity, to implement signature by a biometric technique online. Many works have been developed to improve this technique. They explain the basis for online signature verification. In this type of biometric identification testing, it is compared while the user is drawing on the screen to be matching to the one stored, and that the way to make such signature matches to what was done and recorded in the initial registration procedure by the registered user. To this end, various parameters are measured when making a signature, such as writing speed, pressure or angle of the pen at each point in time when the signature is done, among other features. These signatures can be performed in a special screen that collects and analyzes all necessary signals for analysis or on paper if the pen with which the firm is able to measure the signals described above and send them to a server where you perform the analysis and the signature verification. In patent MX2007007539 collects a system implementing the biometric authentication using an electronic signature. This system includes an interface to a computer capable of storing the movement of a cursor on a computer screen and compared with already stored signature patterns.

The first object of the present invention relates to performing a highly reliable user's authentication capability in a mobile device. Today, there are many applications that can be accessed from a mobile terminal where it is necessary and even critical in many implementations to positively and reliably identify the legitimate user. For years the entrusted all security user's identification on mobile devices are based on a simple solution, that is to type a secret key (PIN) on the phone keypad that the user knew. However, these keys can be easily forgotten, transferred, lost or even counterfeit, so that user authentication is highly compromised. Focusing on the advanced and more secured biometric technique to authenticate a user with a mobile device, are found in US2006286969 and in US2008005575.

In US2006286969 it is proposed to have a remote authentication scheme to authenticate users from a mobile device. The biometric technique used is the voice recognition. The system consists of a mobile phone to send voice samples of an authentication device that connects to a database that stores the identities of mobile phones and voice pattern associated with that phone to make a comparison and check the user is talking on the phone is registered in the system. US2008005575 proposes a method and integrated personal electronic device for authenticating a user on a mobile phone. While the user holds the phone to his ear, a microphone emits a signal near the user's ear and the speaker phone is able to measure the ear's response to this signal. A processor analyzes the response signal and converts it into a signature that uniquely identifies each person and can be used to authenticate. Alternatively various bio-authentication techniques are known as gesture recognition, in which a system is able to detect when a user makes a certain known gesture. Found US2009103780 and WO2009006173 patents related to methods to recognize standard gestures. Patent US2009103780 includes a method for collecting the gestures produced by hand, based on light hand at first by the palm and the back, to get your silhouette associated from various lighting infrared, it proposes a method for collecting various hand movements and identifying a series of gestures previously stored in a database of gestures.

WO2009006173 patent describes a method for detecting the response of an electronically gesture of a user while listening to a speaker using a mobile device, when performing a specific gesture. Related to the idea of recognition of a person by making a gesture is found the patent WO2007134433. It develops a method to authenticate a user when performing an action that manual manipulation of a device such as a mouse. Authentication is to obtain the gesture with the mouse by the user when chasing a target and compare it to the stored pattern of the user when that objective has been pursued previously. Regarding the use of accelerometers in mobile devices, there US2005226468 authentication systems proposed to authenticate the user based on certain biometric sensors must be connected to the mobile device, and verifies that the authentication was successful based on a accelerometer that collects data on how to get the user's device, ensuring it is not a machine trying to cheat the system.

Also, in US2009030350 discloses a method and a system for analyzing patterns gaits is of a subject by measuring the acceleration of the head in the vertical direction while walking. It uses an accelerometer that is placed on the user's head. The analysis includes the creation of a signature from the acceleration data when a user walks. In another invention the prior art also proposes the use of the patterns obtained by realizing the user gesture for generation or release of a cryptographic key. In this connection, patents found DE102005010698 and KR749380-B1. DE102005010698 describes the construction of a cryptographic key for secure communication independent from the fingerprint. It proposes to use that key to communication demand TV with pay per view applications, child protection or age verification.

KR749380-B1 describes a method to generate a key from a biometric characteristic that does not change with time as the iris. The biometric information is received and preprocessed, extracted some values and associated cryptographic key is obtained by grouping the values. The clustering error is corrected using a block of Reed-Solomon code. The obtained key can be applied to any cryptographic system.

Consequently, it is desirable to have a highly reliable mobile platform based medical health record personal data depository, highly protected by enabling access to the user's medical data depository, through a device integrated biometric recognition and authentication module, as will be further described in the present invention, to avoid the drawbacks existing in the previous methods, mobile devices and systems as of the present state of the art. The present invention solution is intended to perform a biometric authentication which brings and combines together the two general characteristics of biometric authentication: the physical characteristics and behavior. Therefore there is also a need in the art to have a mobile communication device that includes modules and methods for high reliability and easy to use way of authenticating a user of the mobile device, and locking or unlocking its communication functions and data storage access capabilities in a case of negative or a positive authentication, that does not suffer from the disadvantages of traditional characters strings based password protection systems and the disadvantages of prior art systems and biometric solutions such as in Shin's or O'Connor's.

Regarding to terminology used in this document portable communication equipment, FASO referred to herein as a “mobile radio terminal”, includes all equipment such as mobile phones, pagers, communicators, Notepads Notebooks and alike, e.g., electronic organizers, personal digital assistants (PDAs), smart phones or the like, It should also be appreciated that many of the elements discussed in this specification, whether referred to as a “system” a “module” a “circuit” or similar, may be implemented in hardware (circuits), or a processor executing software code, or a combination of a hardware circuit and a processor executing code. As such, the term circuit as used throughout this specification is intended to encompass a hardware circuit (whether discrete elements or an integrated circuit block), a processor executing code, or a combination of a hardware circuit and a processor executing code, or other combinations of the above known to those skilled in the art.

### SUMMARY OF THE INVENTION

The following embodiments and aspects thereof are described and illustrated in conjunction with the invention devices, methods and systems, which are meant to be exemplary and illustrative, not limiting in scope. In various embodiments, one or more of the above-described limitations and emerging modern user's growing mobile devices daily secured use needs, have been solved, reduced or eliminated, while other embodiments are directed to other advantageous or improvements of securely managing and updating a modern user personal and private medical data comprehensive depository.

The core of the present invention is an advanced and highly reliable new approach to have and manage user's private personal medical data depository and other health management documentation storage on the present invention dedicated integrated mobile devices while highly protecting the invention mobile device stored medical data depository against intruders, hackers and mal use. The invention device has an is integrated highly reliable user's authentication module, analyzing the user's at least two biometric sensors measure output when user's authentication is done, while the user is holding the mobile device and operating in tandem the biometric sensors measurement on his relevant human body parts and on monitoring his unique human behavior parameters.

One of the main objects of the present invention proposes the creation of a positive and highly reliable and secured user's bio-authentication by implementing an advanced multi-sensors output results analysis fusion by an advanced set of algorithms, done through a dedicated computer SW that is processing the measured outputs of at least two parallel biological and physiological sensors and measuring their output in tandem and provide the final authentication results under a weighted decision factor to create a more reliable user's combined authentication means.

Two of the preferred embodiments of the present invention biometric identification and authentication means and methods may be the user's face recognition and in a second biological user's recognition channel it is done by imaging and analyzing the image of the user's hand—both palm and fingers morphological pattern. In another novel embodiment of the present invention the image of the palm may include also the detection and analysis of the image of the veins and minor blood vessels seen on the palm surface image while imaging the palm with an IR sensitive camera sensor and illuminating the palm with an near IR illumination source, such as a high intensity IR LED. In another user authentication mean may be by a human movement or gesture pattern, while the mobile device is intentionally moved in the air wherein held in the user's hand in a 3D personalized movement pattern that identifies a user, taking into account that this gesture will only be known by the user and also that physical characteristics, it will perform differently to other people who might try to repeat the gesture. Focusing on technical status related to the present invention, it should be noted that performing gestures to biometrically authenticate a person on a mobile device using 3D in-air gestures measured with an accelerometer is novel.

A first aspect of the present invention comprises a mobile electronic device, which enables a user to authenticate himself through the parallel in tandem operation of the present invention mobile electronic device internal integrated set of biological sensors capabilities and then to enable a function of the mobile electronic device using its internal CPU module to differentiate between the authenticated legitimate user and a none authenticated none legitimate user by analyzing and detecting the users personal unique biometric sensors output measurement such as the user's face pattern image, the user palm and fingers image analysis and the user's personalized movement sequence, while 3D moving the mobile electronic device in the air.

The invention integrated mobile electronic device comprises a 3D acceleration measurement module generating an acceleration signal representing the user hand motion in space while holding and uplifting the mobile electronic device. A lock/unlock circuit enables operation of at least one function of the mobile electronic device in response to the measured 3D acceleration signal indicating that the user holding the mobile device hand motion pattern deviates from pre-recorded reference original owners hand motion uplifting movement signal data, while holding and uplifting the mobile device by more than a predetermined threshold.

The lock/unlock circuit may further comprise an integration module and an executable authentication process module. The integration module integrates the acceleration signal with respect to time to generate a velocity signal and a displacement signal. The executable authentication process: i) compares a representation of the displacement signal and the velocity signal, with or without the acceleration measured signal, to the reference motion data. The reference motion data comprising reference displace; and ii) enables operation of at least one function of the mobile electronic device if the representation of the displacement signal and the velocity signal and the acceleration signal data deviate from the reference displacement data and velocity data and the measured acceleration data by more than a predetermined threshold. The reference motion data may also represents the device legitimate user's simple three dimensional gesture movements in space and the user motion represents the device user moving the electronic device in the same simple three dimensional gesture.

In one preferred embodiment of the present invention device there is an integrated mobile personal electronic device, comprising: a. a communication device equipped with a cellular modem, the communication device configured to receive and transmit a user's medical data to and from a plurality of medical data suppliers; b. an electronic add-on sleeve attachable to the communication device thereby forming together a single composite device, the sleeve configured to is securely store and manage the user's medical and health management data; and c. wherein the sleeve comprises a module for activating a lockable set of functions for inhibiting access to said sleeve content.

In another preferred embodiment of the present invention device, the electronic sleeve module of the integrated mobile device is further comprising: a. at least one CPU; b. a solid state, large data capacity, secured memory module; c. a RAM unit; and d. at least two sensor modules, the sensor modules are selected from the sensors group including at least biometric and physiological sensors, thereby to measure and generate the user's authentication by implementing and processing said user individual biometric and physiological parameters.

In another preferred embodiment of the present invention PHR management system the PHR management system is combined of a plurality of remotely distributed integrated mobile personal devices, each containing a secured personal health and medical records storage and data management module, each such integrated mobile personal device is combined of a mobile communication device operating together with an attached mobile electronic add-on sleeve device, the sleeve functioning as a private secure user bio-authentication and medical records storage and management platform, while each of said mobile communication devices communicating with at least one remote medical services provider, the system comprising: a. a system gateway server operating as said system manager for managing and updating ID data of the system plurality of PHR data users integrated mobile personal devices and for securely communicating through the cellular networks and the internet communication infrastructure with each of the plurality of PHR data users integrated mobile personal devices and with the at least one PHR data remote medical services provider serving said PHR data users; b. a memory sub-system connected to the system gateway server to store updated ID data of the integrated mobile personal devices and any required associated user data of each of the plurality of the integrated mobile personal devices users; c. a plurality of integrated personal mobile devices units, each of said integrated mobile devices being associated with a unique user, each unique ID data of said mobile devices being registered with said system gateway server and wherein the ID data file of each unique ID data of the mobile devices being stored in said memory sub-system; d. the gateway server has a plurality of registered medical emergency centers and a plurality of registered medical and health related service providers, all in secured communication through the internet network with said gateway server, the plurality of health service providers comprising at least one of the providers group including health insurance companies, clinics, hospitals, medical imaging institutes, private clinics, government health institutes and municipal health services; e. wherein the gateway server creates access and creates a communication link with any of the selected system registered health service providers in order to get and enable the creation of a direct data access and direct communication link between the selected service provider and a selected specific user's integrated personal mobile device and exchanging and updating personal health management related PHR data and documentation files resident on said user integrated personal mobile device and wherein said access creates a two-way data transfer enabled between any of said system selected user's personal data and documentation files stored by said registered service provider and said user integrated personal mobile device PHR data storage; and f. wherein said communication link is enabled only after positive authentication of the unique user is created by the user's integrated mobile personal device embedded user's bio-authentication capabilities.

Unless otherwise defined, all technical and/or scientific terms used herein have the same meaning as commonly understood by one of ordinary skill in the art to which the invention pertains. Although methods and systems similar or equivalent to those described herein can be used in the practice or testing of embodiments of the invention, exemplary methods and/or systems are described below. In case of conflict, the patent specification, including definitions, will control. In addition, the materials, methods, systems and examples herein are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 is an illustration of a schematic block diagram of one possible embodiment of the present invention integrated mobile personal electronic device configuration, wherein it has combined configuration comprising the invention electronic sleeve shape add-on device, that includes an advanced SOC type microprocessor module, a large storage capacity Flash memory preferably in the shape of a SD card, a user authentication module, an acceleration detectors module, a tilt detectors module, an audio microphone module, two cameras and related image processing module. The is integrated device may include a multi sensors module to measure and prepare for transmission of several physiological measurements of the user carrying the invention device. It also demonstrates the attached second invention mobile device combining mobile communication module that includes the invention personal electronic device implemented and used display and touch screen interaction module and an RF cellular communication module and a GPS location module.

FIG. 2 is a schematic illustration of a state machine in the form of a flowchart, wherein the flowchart is representing the initials stages of the device application mode of preparation stage and its sensors activation events, prior to the initiation of the authentication process, possibly followed by the activation a user PHR interaction screen, done through by the present invention mobile device display module, according to one possible embodiment of the present invention.

FIG. 3 is a schematic illustration of a state machine in the form of a flowchart, wherein the flowchart is representing device user's authentication execution, to be then followed in cases of a legal and an illegitimate user, according to one possible embodiment of the present invention.

FIG. 4 is a schematic illustration of an example of a computerized PHR management system, according to one embodiment the present invention system.

FIG. 5 is a schematic state machine in the format of a flow-chart illustration of one possible embodiment of the present invention regarding the invention system operation. It is demonstrating a user interaction process with a requested group of the system registered service providers, the described process starts when the user has reached a stage in the invention system user's access creation process, wherein at this stage the user has received full access confirmation through his mobile integrated device, wherein the user can get access to medical service providers data access through the system servers to the invention PHR management system resources about his private medical health related updated data.

### DETAILED DESCRIPTION OF THE INVENTION

The present invention, in some embodiments thereof, relates to dedicated integrated new mobile devices that are personal to their single owner, wherein the devices are serving their owners for PHR management and for updating personal mobile health & medical data management and, more particularly, but not exclusively, to methods, a device and a systems to manage and conduct mobile devices operational permit through bio authentication and the following execution of various alarms and notifications in case of a non-legitimate user authentication failure.

Before explaining some embodiments of the invention in details, it is to be understood that the invention is not necessarily limited in its application to the details of construction and the arrangement of the components and/or methods set forth in the following description and/or illustrated in the drawings and/or the examples. The invention is capable of other embodiments or of being practiced or carried out in various ways.

As will be appreciated by one skilled in the art, aspects of the present invention may be embodied as a device, a system, a method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, integrated personal electronic device, or device, or any suitable combination of the foregoing. More specific examples (a non-exhaustive list) of the computer readable storage medium would include the following: an electrical connection having one or more wires, a hard disk, a random access solid state memory (RAM), a read-only memory (ROM), an erasable programmable read-only memory (EPROM or Flash Memory), an optical fiber, an optical storage device, a magnetic storage device, or any suitable combination of the foregoing. In the context of this document, a computer readable storage medium may be any tangible medium that can contain, or store a program for use by or in connection with an instruction execution system, integrated personal electronic device, or device.

A computer readable signal medium may include a propagated data signal with computer readable program code embodied therein, for example, in baseband or as part of a carrier wave. Such a propagated signal may take any of a variety of forms, including, but not limited to electronic, electro-magnetic, optical, or any suitable combination thereof. A computer readable signal medium may be any computer readable medium that is not a computer readable storage medium and that can communicate, propagate, or transport a program for use by or in connection with an instruction execution system, integrated personal electronic device, or device.

Program code embodied on a computer readable medium may be transmitted using any appropriate medium, including but not limited to wireless, wire-line, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's smartphone, partly on the user's smartphone, as a stand-alone software package on the user electronic sleeve shaped add-on computerized device, partly on the user's smartphone and partly on a remote computer, or entirely on the remote computer or server. In the latter scenario, the remote computer may be connected to the user's mobile device through any type of network, or the connection may be made to an external computer (for example, through the Internet using an Internet Service Provider, or through a cellular service provider).

Aspects of the present invention are described below with reference to flowchart illustrations and/or block diagrams of devices, methods, systems and computer program products according to different embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a smartphone, on an electronic sleeve shaped smartphone add-on computerized device, a notepad, a laptop, a special purpose computer, or other programmable data processing integrated personal electronic device to produce a machine, such that the instructions, which execute via the processor of the computer or other programmable data processing integrated personal electronic device, create means for implementing the functions/acts specified in the flowchart and/or block diagram block or blocks.

These computer program instructions may also be stored in a computer readable medium that can direct a computer, other programmable data processing integrated personal electronic device, or other devices to function in a particular manner, such that the instructions stored in the computer readable medium produce an article of manufacture including instructions which implement the function/act specified in the flowchart and/or block diagram block or blocks.

The computer program instructions may also be loaded onto a smartphone a mobile or portable computerized device, other programmable data processing integrated personal electronic device, or other devices to cause a series of operational steps to be performed on the computer, other programmable integrated personal electronic device or other devices to produce a computer implemented process such that the instructions which execute on the computer or other programmable integrated personal electronic device provide processes for implementing the functions/acts specified in the flowchart and/or block diagram block or blocks.

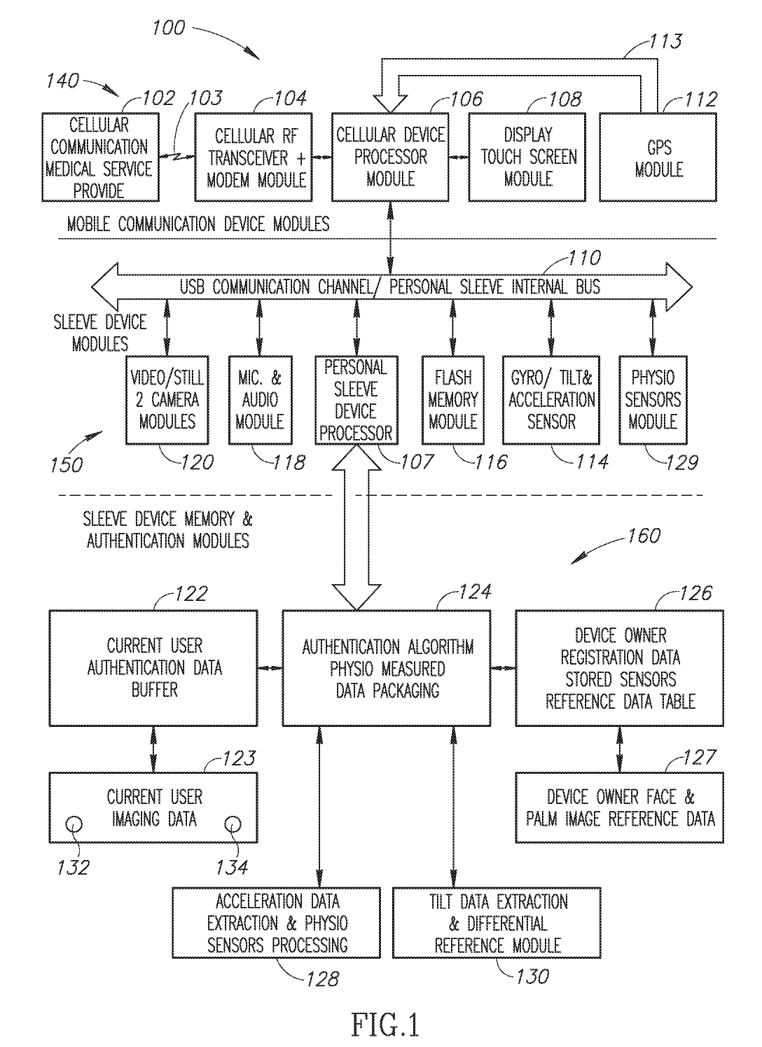

Reference is now made to FIG. 1, which is an illustration of an example of a computerized mobile electronic device hardware and software content and related configuration, according to the present invention. Mobile personal electronic device 100 is representation of an exemplary present art mobile personal electronic device geared for serving as a single user medical records data depository storage, updating and management tool with a built-in user's bio authentication capabilities based on at least two device integrated biometric sensors output analysis of the user, done while the user holding in his palm the integrated personal mobile electronic device 100. The present invention integrated mobile device 100 is communicating through the cellular and internet communicating networks with a typical medical service provider 102 through the cellular wireless networks, as required to operate some of the dedicated functions related to the present invention mobile personal electronic device medical depository and health related data management functions. Cellular RF transceiver and Modem module 104 is representing the mobile device wireless cellular or direct link module that supports and enables the device data communication with the medical service provider 102. The mobile communication device processor 106 is controlling the entire mobile communication device various functions of communication, graphic& alphanumeric display control and the user's flat screen graphic management and processing capabilities. The electronic add-on sleeve processor 107 is managing the sampling and the processing of the device movement sensors 114 data, the imaging 120 and voice sensors 118 and an internal/external add-on user's physiological parameters measurement and signal processing sensors module 129. A part of these sensors are required to support the execution of user's authentication program module 124, based on the selection of at least two bio sensors modules of the invention electronic sleeve 150, selecting between the activation of the user gesture movements data from 130 and 127, the face 132 and the palm 134 real time cameras sampled images combining the imaging recognition data module 123 and user's voice pickup sensor and voice processing 118 sampled data. The display module 108 is a combination of a graphic/image display screen and a touch sensitive screen to support the user's various interactions and the display of the interactions results with the mobile personal electronic device 100 through the mobile communication device 140. The electronic sleeve shaped electronic device central internal data communication bus and USB interface based communication channel 110 operating within the communication device 150, supports the needs to transfer data and commands between the various modules of the integrated personal mobile device 100. Module 120 is the mobile device add-on sleeve resident internal still imaging module, 120 including one or more electronic camera units equipped with aided LED visible and near infrared active illumination that support the imaging of the device user face and/or palm, in order to document and authenticate the user according to his face and/or palm & fingers details pattern according to some embodiments of the present invention. Audio module 118 is including at least one microphone that enables the voice identification of the user as one of the selected biometric sensors required for the potential execution options of the user's authentication, based on the user voice personal characteristics analysis. Flash memory module 116 is at least one of solid state memory modules resident within the electronic sleeve shaped add-on device 150 that holds the operational software of the sleeve shaped add-on device, as well as the functional software modules 122, 124 and 126 that support the invention integrated mobile device 100 requirements to function as PHR medical data depository of a single user. Tilt and acceleration 3D sensors module 114 is a unit resident within the invention integrated mobile device 100 electronic sleeve add-on device 150 that measures the linear acceleration on the three orthogonal axis of the device and the 3D tilt angles of the sleeve device 150 in space. GPS module 112 is another important built in sensor resident within the present invention mobile communication device 140, wherein the GPS world coordinated device dynamic poison reading is fed through communication channel 113 to cellular device processor module 106 so to enable the allocation of the device in case of a user problematic medical situation emergency cases.

Authentication data buffer module 122 which is a part of the electronic add-on sleeve device 150 memory and authentication sub-section 160, is a secured memory buffer containing the device biometric sensors sampled data of the mobile personal electronic device user's authentication reference data, as the stored digital converted output of the various sampled mobile electronic sleeve shaped device 150 resident biometric sensors, wherein the biometric data is collected and stored during the user first and initial enrollment registration process. The data buffer module 122 is also connected with sub-module 123 that samples and stores the current user face and palm images digitized data, to further use it as the user's additional channels of bio authentication sources, according to the present invention authentication embodiments. Module 124 is the central SW module in the present invention electronic sleeve device 150 managing the selection of optimal process for selection, choosing and executing the optimal residing authentication algorithm, choosing the optimized one of several authentication algorithms and significant user identification sources options. The 124 module does the analysis the user's biometric sensors 120, 118, 130 and 127 modules output. The 124 module also creates the improved quality and reliability of the authentication process of the integrated mobile electronic device 100 while fusing together the user's measured biometric sensors output, wherein the method is implementing into the authentication process the user hand in air 3D gesture, the user's face pattern and the palm recognition imaging data, as the first, the second and the third sources of the user's bio personal data, thus enabling an optimal quality authentication process, combining gesture, face and palm personal bio data.

Module 124 has in one of the invention embodiment another additional set of functions for execution in the cases that the authentication process of the current device holder is indicating a failure, which is a non-authenticated user case. In such a case the module 124 is creating a series of preprogrammed alarm functions, creating audio alarm set of signals on the audio module 118 and displaying visual eye attracting flashing images through the display module 108. In parallel alarm data is sent from the invention mobile device to a remote cellular service provider and through it to a set of the users who are the device owner group of pre-selected piers to notify them on the event of theft or loss of said device 100 and the location of the theft, as it is constantly read and transmitted via data link 113 with the invention mobile device 100 location data created by the GPS module 112. Software module 126 is storing and managing legitimate user reference registration data, as required by present invention integrated mobile personal device 100, while managing the registration procedure of the legitimate user prepared and stored by module 126 to serve as the reference set of data while compared to the current user biologic sensors measured and processed authentication data. A sub module connected, functioning with and used by module 126. SW module 127 stores and manages the legitimate device owner face and palm pattern reference data created at enrollment session, The recorded reference imaging data including the legitimate user face and palm pattern recognition parameters and also may potentially store and manage the registered user's selected small group of piers (close friends and family) with their face recognition data to avoid false operation of the device alarm functions when one of the legitimate user's piers is by mistake lifting and holding the invention 100 protected mobile device. Module 128 is a SW module that manages the extraction and processing of the sampled output of a set of physiological sensors 129. Module 128 processed and stored physiological data package selected out of the total output of all the user's selected output of all physiological daily measured parameters by the sensors module 129. These sensors module may include one or more sensors selected from the sensors group including at least; a heart bit rate measurement sensor, a blood oxygen (O₂) % content sensor, an EKG measurement sensor, an EEG measurement sensor, a blood sugar content measurement sensor, a body temperature measurement sensor, a blood pressure measurement sensor, a GSR skin perspiration measurement sensor, a video camera sensor for user's face image expressions and skin color status analysis, an eye Iris recognition imaging sensor, a body heat measurement indicator, an electro-dermal activity indicator and a physical or emotional stress sensing indicator.

The measured physiological sensors output are then transferred within said sleeve device 150 to the physio data packaging module 124 and then the selected user's measured physiological set of parameters is then fed to the communication device 140 to be securely transmitted through cellular link 103 through modem 104 to the selected remote medical staff, resident in the remote medical service providers 102. Also module 128 is used for processing of the integration algorithms on the acceleration measured device 114 results data, in order to achieve data related to the device velocity and position in space, based on the acceleration data one time for speed/velocity and two times integration for position calculation results. Module 130 is a SW module that manages the extraction of the sampled gyro based tilt measurement 3D set of sensors 114 and also in processing derivatives algorithms on the tilt angles measured device data results, in order to achieve data related to the device angular velocity and angular acceleration in space, based on the measured 3D tilt angles data, one time and two times derivatives calculation results.

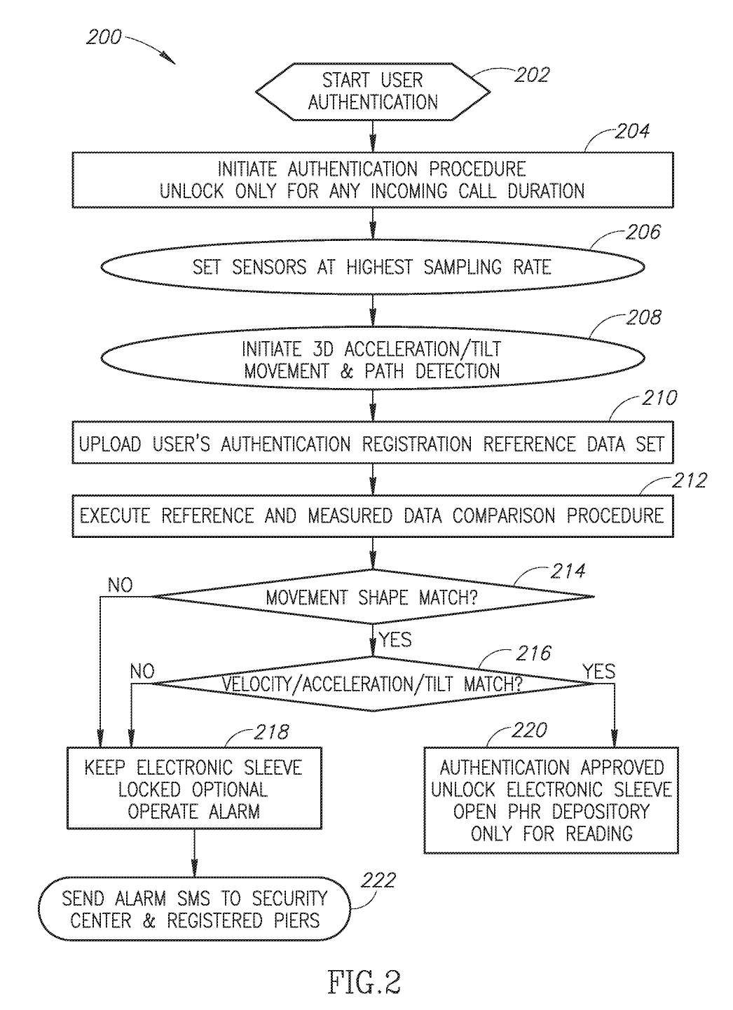

Reference is now made to FIG. 2, which is a schematic illustration of a state machine wherein states reflect actions and transition arrows relate to external triggers which are performed with regard to a certain layout, according to one embodiment of the present invention, wherein this state machine is demonstrating the different change of states of the present invention PHR storage and management tasks done during authentication process by the invention secured integrated mobile personal device 100, and wherein the flowchart 200 is representing the user's single biometric sensor based activated standalone authentication process, with an optional alarm activation phase of the invention combined mobile device. This possible embodiment covers the user's standalone simplified authentication process use case of the medical PHR depository management SW driven stages and the implementation of only one of its alternative original user selectable integrated biometric sensors for executing the authentication process. In this embodiment, the user is activating the optional in-air movement measuring sensors, according to one possible embodiment of the present invention. Stage 202 is representing the start of the authentication process of the invention integrated mobile personal device user who is uplifting and holding the device. Stage 204 is representing a state that is requiring the device to enter into the authentication process and in parallel to block the mobile communication device of the present invention combined mobile personal electronic device from making and executing any outside calls or external data communication such as emails/SMS and internet data exchange and enables for the user convenience only the receipt of incoming calls until finalizing the authentication process. Then after authentication completion, either clearing or accepting the current user as the legitimate owner enabling him full access to his mobile sleeve add-on personal device PHR secured data depository. In stage 206 the device program controls the related execution of the sleeve processor 107 shifting the electronic sleeve add-on device to the highest possible movement sensors sampling rate of typically, but not exclusively to 20 samples/sec, thus enabling an optimized movement pattern detection and sensing capability, and using the required higher battery power consumption only for the short time duration that is required to execute the current user several authentication process stages. In stage 210 the device program is uploading from the device memory the stored user's enrollment measured device owner recorded sampled gesture comprising the user in-air movement pattern sampled data, a file that has been done and stored in the invention sleeve add-on device memory in the initial registration phase of the device owner, as required to initiate the PHR depository protection entire procedure in the present invention integrated mobile personal device. Following to uploading from the sleeve device memory the device stored owner's recorded registration gesture reference sampled data done in stage 210, then in the following stage 212 the sleeve device is executing the comparison of the sampled stored owner's in-air gesture data to the sampled present user measured sensor's movement samples of the mobile device in-air gesture data. If the measured 3D shape of the present user device in-air movement pattern in space is matching the software based reconstructed device in-air movement pattern shape in space of the device owners original registration data and if they match each other above a pre-defined threshold level, then the device authentication phase program moves to stage 216 for another higher level comparison and matching test phase. In stage 216, the device is executing at least one out of three measured movement parameters comparisons to the stored device owner registration related data. The stage 216 comparison activities is comparing the uplifted device sampled 3D movement data sequence in the authentication session, implementing one or more of the device acceleration, velocity and tilt angles parameters and their associated sampled data sequence change in time during the measured user gesture time—to the identical set of data samples done by the legitimate device owner and recorded on the device during the initial user's registration phase, If the matching score is also high above a predefined threshold level, then the device state machine is moving to stage 220 where the device has fully authenticated the present user and identifies him as the device original and legitimate registered user and then it opens the user access the invention electronic sleeve PHR depository data content as long as the authenticated user is holding the device until later when the user exits the PHR data depository the device is going back to stage 202. When the comparison results of decision making stage 216 show negative match results between the reference device 3D movement shape data and the present user device holding hand movement shape data, then the state machine moves to stage 218 in which user's access to the PHR data depository is denied, a relevant textual message appears on the integrated mobile personal screen while an optional user selected alarm activation stage, when the invention integrated mobile personal device is activating a preprogrammed high level sound alarm selected by the user while in parallel also a visual alarm displayed session is running on the device screen with visual alarm graphics and textual notes. Further to stage 218 when the device has activated the alarm, the device operational state machine is moving to stage 220 where a SMS alarm notification is generated by the device 100 and sent through the cellular networks 103 to a list of cellular numbers of registered user's piers and relatives of the notifying them on the device theft or loss and sending to them the present GPS measured device position coordinated, or Google location based services of Wi-Fi hot-points triangulation based reading, covering the detected present location of the device and an optional image of the device present carrier/user, that is made automatically in a sequence of still images made by the device embedded camera, activated only when stage 218 is activated.

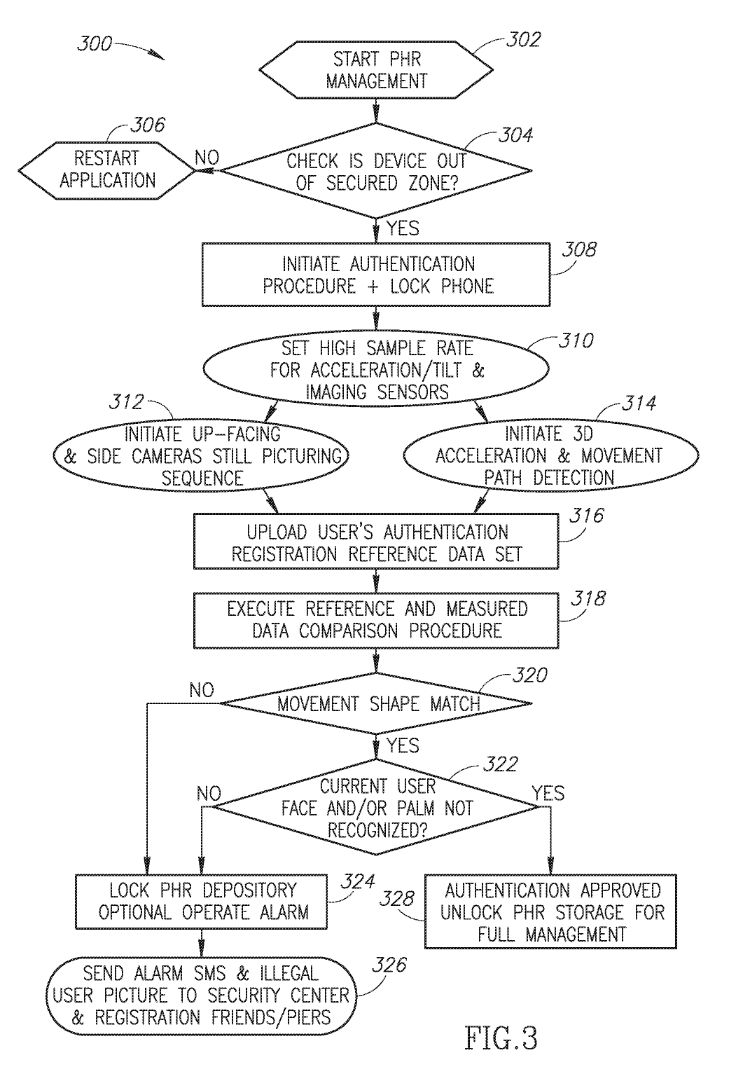

Reference is now made to FIG. 3 is a schematic illustration of a state machine wherein states reflect actions and transition arrows relate to external triggers which are performed with regard to a certain layout, according to another embodiment of the present invention, wherein this state machine is demonstrating the different change states of the present invention secured integrated mobile personal device 100, and wherein the flowchart 300 is representing the user's authentication process and possible optional selectable alarm activation phase of the device when the authentication process is initiated where the device is authentication is done by operating in tandem two biometric sensors according to the present invention full scale authentication method. Stage 304 is the starting point in the activation of the state machine of the SW, activating the device. In stage 304 the device is checking its position coordinates by using its integrated GPS or reading the Google positioning services data to analyze and define if its location is in a safe zone or not. This safe location state is related to being in protected and trusted pre-defined user's/device location zone, like home, family, or work place. If location is found to be in a safe zone it moves to stage 306 were the mobile device is safe open for all available device uses and waits for change in location or basic activation of authentication by the user according to the simplified authentication process described in FIG. 2. If the location assessment in 304 finds the device to be in a non-secured zone, then it moves to stage 308 where automatically it starts requesting the user/holder to do a double sensor based authentication based action and in parallel it is locking and blocking the mobile device communication and other operational capabilities by the automatically firing of the procedure described in FIG. 2. In this device operational and functional present invention double sensors based authentication embodiment, the device is also automatically activating in stage 312 also the face recognition sensor camera embedded within the invention sleeve device case facing the suspected user's face. To enable higher accuracy measurement of the device accelerometers and gyro/tilt position sensors, the invention integrated mobile device is then upgrading its sampling rate to the highest level possible without consuming too much energy for the sleeve device battery. Typically such sampling rate can be chosen to be 20 samples/sec or higher. In stage 310, the device is initiating the highest sampling rate of the gyro based or other angular tilt sensor fast sampling mode and in parallel in stage 314, the device is initiating its fast acceleration measurement-sampling mode using its integrated accelerometer sensor.

In parallel in stage 312 the device is initiation a sequence of exposures of the integrated camera imaging the user in order to accumulated maximum visual data on the face recognition of the evaluated user, alternatively the user can choose the side camera for imaging the palm morphological pattern of his extended hand palm facing the side camera. In stage 316, the state machine shows the operation wherein the device SW is uploading the original user movement and face/palm measured data recorded and stored during the initial stage of the legitimate device owner/user first enrolment and registration procedure. In stage 318 the state machine shows the actual authentication phase execution by comparing the movement results of the user hand holding the device to the stored user's hand movement recorded data collected during the user's initial enrolment stage and in parallel comparing the main facial features of the photographed user to those recorded and securely stored in the sleeve device secured memory during enrolment. Stage 320 is a decision stage of the state machine by comparing the 3D movement graph shape of the user to the shape done and recorded in the enrollment initial phase. If comparison results show a bigger than a predefined threshold level deviation between the two present and past recorded 3D movements graphs, then the state machines moves to stage 324 when device is notifying present user on authentication failure and possibly activating a user optional selectable full alarm display and sound generation. If movement shape of the enrolment data file and the present user movement data file are matching above a predefined threshold, then the state machine is moving to stage 322 wherein comparing the measured face recognition main features of the user's photographed face between the present user imaged face and the registered set of user's original enrollment process face image main features recorded in the face recognition process. If the results show no match, the state machine moves to stage 324 notify the present device-holding user of non-authentication results and activates an optional prior original user selectable alarm mode as well. If comparison done on stage 322 show good match above a predefined SW driven matching threshold, then the invention electronic sleeve device CPU moves the state machine to state 328, wherein the device SW stating to the present combined mobile device holder that full user's authentication is approved and the user can use the combined mobile personal device for PHR depository content maintenance and updating functionality for, all its available operational functions. Stage 326 is activated as a final conclusive stage of a non-legitimate user with no authentication final approval stage, wherein the user is defined by the device as a non-legitimate user trying to still or use the device with no authorization. In such a case optional stage 326 the device sends an alarm and the face pictures of the suspected non authorized user by a text/image message (SMS or WhatsApp message) to a dedicated security entity and/or to remote multiple users and in parallel sending a similar alarm, suspect thief images and theft warning messages to pre-recorded contact details of friends of the legitimate device owner.

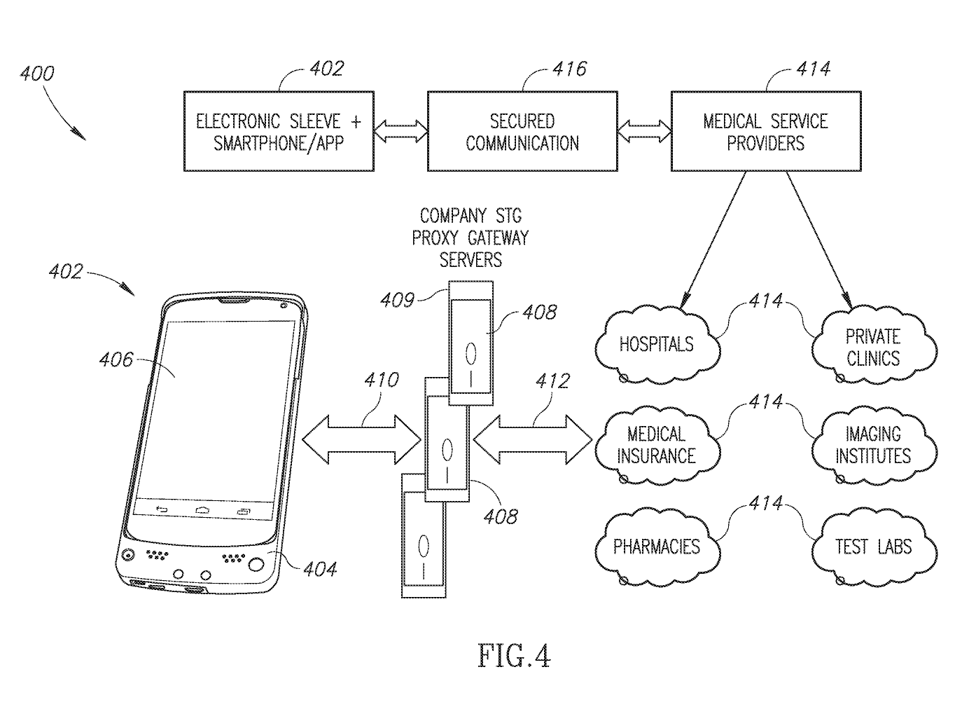

Reference is now made to FIG. 4, which is an illustration of an example of a computerized PHR management system, according to one embodiment the present invention system. The present invention PHR management system 400 is combined of a plurality of remotely distributed number of integrated mobile personal devices 402, each such mobile personal device 402 is containing a secured personal health records (PHR) storage and management data module, each of the present invention integrated mobile personal device is constructed of a mobile communication device 406, in most cases it is a modern flat screen smartphone or a palm computer and communication device, such as an IPad, both unit are assembled together as a unified carried mobile unit together with the invention attached mobile add-on electronic sleeve device 404. The mobile sleeve device 404 is functioning both as a private user bio-authentication means and as a medical and health management records secured storage and management platform. Each of the invention integrated mobile personal devices 402 is securely communicating through an encrypted communication protocol over the Internet 416 with at least one remote medical services provider 414 through the invention integrated device 402 mobile integrated communication modem.

The invention multi-users PHR management system 400 is comprising of the following main elements: a; a system gateway server 408 operating as the invention PHR management system 400 manager, for managing and updating the ID data of the system 400 plurality of PHR data users mobile devices 402 and for securely communicating 410 with each of the plurality of PHR data users mobile devices 402 and in parallel securely communicating 412 with a plurality of PHR data suppliers remote medical services providers 414; b: a memory sub-system 409 connected to the system gateway servers 408 to store updated ID data of the mobile electronic sleeve device 404 and any required associated user data of each of the plurality of the mobile devices 402 users; c: a plurality of personal mobile devices units 402, each of these mobile devices 402 is being associated with a unique user, each unique ID data of the mobile devices sleeve units 404 is registered with the system gateway server 408 and wherein the ID data file of each unique ID characterizing number of the mobile devices 404 is stored in the servers 408 memory sub-system 409; d: a plurality of the gateway servers 408 are communicating with registered medical emergency centers and with a plurality of registered medical and health related service providers 414, these PHR data suppliers are in communication through the internet network with the gateway servers 408. The plurality of health service providers 414 include health insurance companies, medical testing clinics, hospitals, medical imaging institutes, private clinics, medical rehabilitation and specializing clinics, government health institutes and municipal health services and pharmacies; and e. wherein the gateway servers 408 access and creates a communication link with the any of the system registered health service providers to get access to their pre-registered users' personal health management related medical and administrative data and documentation files and wherein the access to any of the system registered users' personal data and documentation files generated, processed and stored by the registered medical data generating service providers 414, the access to transfer of the selected user data is only enabled after positive authentication of each of the system registered unique users by their integrated mobile personal devices 402 supported and executed by identifying the users' personalized identification data, associated by the system servers 408 with the registered owners of the integrated mobile personal devices 402 through the identification of their composing sleeves 404 unique production serial number. These electronic sleeve shaped add-on mobile devices are a critical PHR data management component in each one of the system 400 user's mobile personal mobile devices 402.

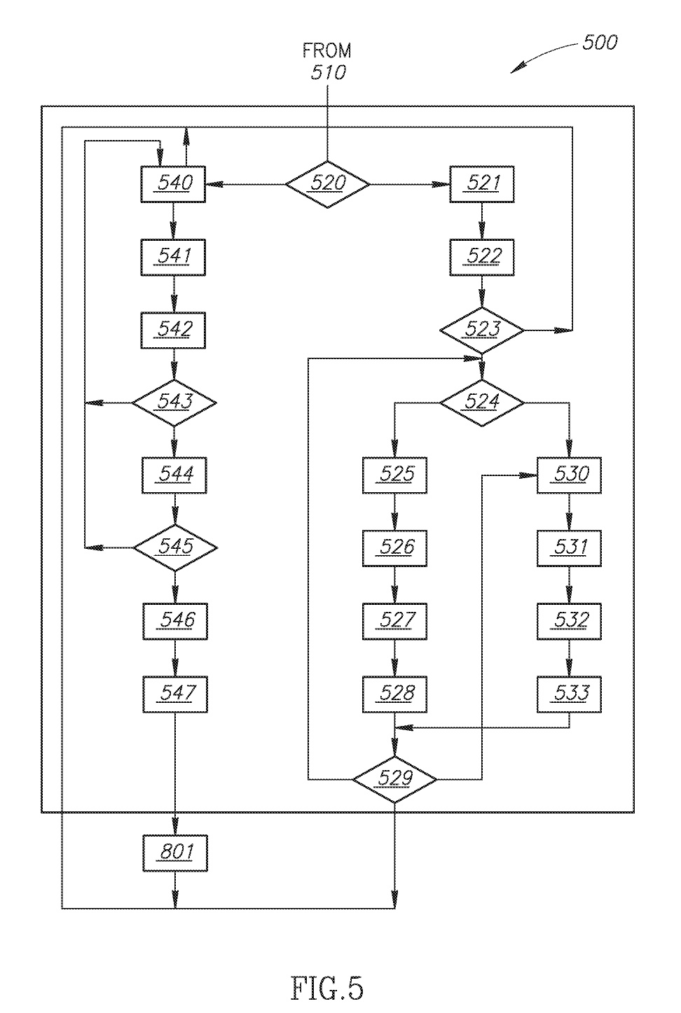

Reference is now made to FIG. 5. is a flowchart of one embodiment of a possible flow of the process steps, generally designated 500, associated with the use of the invention integrated mobile personal electronic device 402 geared for the user PHR depository and patient PHR related data full management and updating needs, while a user is establishing a connection with the invention system 400 by applying the invention integrated personal electronic devices 402 to be first authenticated as shown in FIG. 3 and then get access to the system servers 408 before getting direct bi-directional communication access permission and data exchange capability with the various system 400 registered medical service providers 414.

FIG. 5 is in the form of a flowchart that is describing the process steps associated with the use by each of the system 400 users of the invention integrated mobile personal electronic device 402 and the user interaction with the invention PHR management system 400 after the prior preliminary stages wherein the user is positively authenticated and then getting an approved access to the invention PHR management system gateway server 408. The described process in this flowchart starts from completion of the stage wherein the system 400 user through the personal device 402 is getting secured access through the system servers 408 followed by the user requested data exchange interaction specific medical service provider 414. There is an initial session of communication interaction between the system servers 408 and a specific service provider 414 followed by a user personal device 402 sending to the system servers a specific interest request to get direct access for PHR data exchange from one or more of a specific system registered service provider 414. There are various kinds of system 400 registered medical service providers 414, such as; hospitals, medical clinics, HMOs and other medical entities and services providers, that are required to fulfill the user specific needs for related personal medical data files search, and serving the data communication required for the user's medical records updating and then, if required, also executing the updating process of the user's medical records into his mobile personal electronic device 100 secured storage 116.

It is shown in FIG. 5, in the invention system operation, illustrated as a flowchart of a state machine, demonstrating a user interaction process with the requested service providers, the process starts when the user has reached stage 520 in the invention system user's access creation process 500, as described in FIG. 5. Starting point is stage 510, in process 500, at this stage the user has received full access through the system 400 servers 408 to the invention PHR management system and the user has selected the option in his integrated mobile personal electronic device 402 interaction screen to get access through the system 400 servers 408 to download or upload medical, as well as administrative and billing management data dealing with the user's personal medical records and medical health management and getting secured access through the system to the user's various PHR data files, including also the user's financial and administrative data management and exchange with the system 400 registered medical service providers 414.

In stage 520 of the FIG. 5 described process, the user receives a selectable menu screen requesting him to choose between selected medical insurance companies (HMO) and then he goes to stage 521, or alternatively to choose hospitals, then he goes to stage 540, or to choose a laboratory or a testing clinic 602, then at the end of each choice he goes to the final stage 800, or for ending the process it is done by going back to stage 302 as described in FIG. 3.

In stage 521, the invention PHR management system 400 creates a secured access to the medical insurance company server that the user is a member of, according to the relevant data in the medical records stored in the user's invention integrated personal electronic device 402 memory.

In stage 522, the user's integrated personal electronic device 402 sends to the medical insurance server 414 the user's member username and medical insurance member ID number and if requested also the user's access password to the medical insurance website. In stage 523, the medical insurance company server checks if is the user medical file ID data fits to the ID and user data that was received from the user's integrated personal electronic device 402 through the invention system and continues to stage 524 if positive and returns back to starting point stage 510 if received user's identification information results are not acceptable and consequently the acceptance response is negative.

In stage 524, the system requests the user to select between several options in a menu screen that he gets on through his mobile personal electronic device display. In option 525, the user may choose making a doctor/clinic appointment. In choosing option 530, the user requests all his recent past medical tests results not yet updated to his mobile personal electronic device PHR data memory, otherwise the user is directed back to stage 520 and then stops if no further action item is selected by him.

In stage 525, the user receives a screen from the medical insurance through the invention PHR system fed into his mobile personal electronic device display, requesting the user to select the professional type and the name of the medical doctor he wants to meet. In stage 526, the user gets a screen with the reception open dates and hours for the selected doctor. In stage 527, the user is selecting and approving his best date and time choice for appointment with the selected doctor.

In stage 528, the user receives from the medical insurance computer 414 a final confirmation notice screen with the reception open dates and hours for the selected doctor regarding the user's final approved medical appointment time and day, then the process continues to stage 529. In options selection stage 529 the user receives a menu on his mobile personal electronic device 406 display screen to go back to 524 or to see his past medical tests results for which option selection he goes to 530, or to return through starting point 510 to main menu 520, or to save tests results in stage 501. In stage 530, the user receives on his mobile device screen display 406 a menu screen from the medical insurance server, to select the medical tests results he wants to review.

In stage 531, the user selects the requested tests results and the user's integrated personal electronic device forwards to the user's personal device screen 406, the most updated results the user has in his integrated personal electronic device memory on this specific test, if any. In stage 532, the medical insurance company is server searches its memory records to check if it finds more relevant updated tests data on this user and then it sends the found files to the user's personal mobile device to enable the user to select either to display the results on the device screen or/and to save them in his integrated personal electronic device memory for long term storage. In stage 533, the user selects his choice on the processing of the retrieved specific one or more medical tests results and the system goes back to stage 529, which may connect to the updated PHR data save stage 801.

In stage 540, the user receives a menu screen on his mobile device display 406 to choose a hospital from a list, or to return to stage 520. He then chooses the preferred hospital from the list and then goes to stage 541. In stage 541, the system server 408 contacts the selected hospital via a secured communication channel 416 it has with each of the hospitals in the displayed list and creates an open communication link with the hospital server 414. In Stage 542, the user's personal integrated personal electronic device 402 sends the user's ID data file including full name, ID card number and basic insurance and medical profile details. In stage 543, the selected hospital server checks if it has in its records that the specific user was a hospitalized patient or was treated by the hospital's clinics; if yes, it goes to stage 544, if not, it goes back to stage 540 and creates and automatically fills-in a new patient acceptance entry file document. In stage 544, the user receives a screen with a menu requesting him to choose if he needs a hospital release medical report, medical tests results, or billing and administrative reports and the user selects his choice.

In stage 545, the hospital server searches for the selected data files and goes to 546 if the requested data files were found, and if not, the process goes back to stage 540. In stage 546, the user receives the specific requested information from the hospital on his integrated mobile device screen 406. In stage 547, the user receives a menu screen requesting him to choose where to store the retrieved hospital data in the hospital system memory, wherein the user's personal medical data files are stored, or in the user's personal integrated personal electronic device 402 memory, or if both then it goes to stage 801.

After the new medical files are saved and stored in one or two of the selected storage memories in stage 801, then the process goes back to stage 520, where the user gets a new menu screen to start another medical data processing sequence with the is invention system and with external medical services providers, or goes back to start a new interaction sequence stage 302 with the PHR management system 400, as described in FIG. 3.

1. An integrated mobile personal electronic device, comprising:
a. a communication device equipped with a cellular modem, said communication device configured to receive and transmit a user's medical data to and from a plurality of medical data suppliers;
b. an electronic add-on sleeve attachable to said communication device thereby forming together a single composite device, said sleeve configured to securely store and manage said user's medical and health management data; and
c. wherein said sleeve comprises a module for activating a lockable set of functions for inhibiting access to said sleeve content.

2. The sleeve of claim 1, said sleeve further comprising:
a. at least one CPU;
b. a solid state, large data capacity, secured memory module;
c. a RAM unit; and
d. at least two sensor modules, said sensor modules are selected from the sensors group including at least biometric and physiological sensors, thereby to measure and generate the user's authentication by implementing and processing said user individual biometric and physiological parameters.

3. The sleeve of claim 2, wherein said secure memory module containing at least a secured medical and health management data storage content and additional operational and security management functions of said mobile electronic device.

4. The sleeve of claim 2, wherein in response to said sensors module measured and sampled signal sequence said signal analysis is executed by said sleeve CPU comparing said at least two user's measured sensor modules output signals to the related user's pre-recorded and securely stored under an initial controlled registration procedure of at least identical two user's sensors module authentication reference signals results.

5. The personal electronic device of claim 2, wherein said sleeve is further configured for encrypt and decrypt said stored and managed user's medical and health management data; and
wherein said encryption is done by a dedicated module that resides within said sleeve CPU secured zone and is used to encrypt and decrypt said data content of said user medical records depository stored as encrypted data in said mobile device sleeve memory and the encrypted data of said two biometric sensors also being recorded and stored encrypted for further authentications as the biometric sensors registered user authentication reference data.

6. (canceled)

7. (canceled)

8. The personal electronic device of claim 1, wherein;
a. said lock/unlock set of functions is positively activated and said device sleeve secured memory is unlocked and ready for use, only for a short programmable time duration when said legitimate mobile device user is activating said at least two measured user's biometric data parameters and they are being positively compared with said legitimate user pre-recorded reference data, while results are deviating from each other by less than a predetermined minimal threshold;
b. wherein said electronic add-on sleeve has at least one selected from a group of communication mean, including; a USB wired communication module, an integrated cellular communication modem, a Wi-Fi communication module, a NFC short range communication module and a Bluetooth short to mid-range communication module; and
c. wherein said communication device further containing a touch screen display unit for indicating said device status and displaying its selected data content and for the user's interaction with said data content of said personal mobile electronic device.

9. The personal electronic device of claim 1, wherein at least one of said at least two sensors is a life signs physiological detector module, said life signs detector module being configured to measure and record at least one of said user's life sign user's health indicating parameters; wherein
a. said life signs module comprising one or more sensors selected from the group including at least; a heart bit rate measurement sensor, a blood oxygen (O₂) % content sensor, an EKG measurement sensor, EEG measurement sensor, a blood sugar content measurement sensor, a body temperature measurement sensor, a blood pressure measurement sensor, a GSR skin perspiration measurement sensor, a video camera sensor for user's face image expressions and skin color status analysis an eye Iris recognition imaging sensor, a body heat measurement indicator, an electro-dermal activity indicator and a physical or emotional stress coercion sensing and indicator; and
b. wherein at least one of said plurality of life signs sensors output is recorded and stored in said electronic device memory and upon user's selection or at upon prerecorded selected time duration also sent to external medical service providers sites for said selected sensors output further processing and for user's health related remote management by medical teams.

10. The personal electronic device of claim 1, wherein said at least two measuring biological parameters measurement sensors are selected from the sensors group including at least; a three dimensional device comprising an air-gesture linear acceleration based measuring sensor, a three dimensional mobile device air-gesture angular movement tilt measuring sensor; a face recognition imaging sensor, a hand geometry imaging and analysis sensor, a palm pattern and morphology imaging sensor, a palm veins structure imaging sensor implementing active IR illumination for optimized imaged palm veins networks picture analysis—enabling the veins pattern measuring and analysis assisted by an added IR/visible illumination source, a user fingerprint pattern imaging or ultrasound based sensor, a user's voice analysis based on a voice pickup microphone sensor and a voice processing module, an eye Iris imaging sensor and at least one user's life signs indicating sensor.

11. The integrated mobile personal electronic device of claim 2, wherein the digital output of one type of said sensors user's biological parameter measurement results and said measured at least one additional type of sensor output results, are fused together by using a learning and adaptable dynamically weighted factor fusion algorithm, executed upon said two different types of sensors measured output, in order to enable improved and precise analysis and identification of the exact legitimate user typical personal characteristics while minimizing signals background noise and signal destruction effects and wherein said algorithm is creating a highly reliable user's authentication mechanism to best decide, while comparing to a similar reference prerecorded fused set of said two identical kinds of sampled user's biometric sensors, if to activate said lock/unlock circuit for enabling access to said secured device user's medical data storage, or to enable the operation of one or more functions of the mobile electronic device.

12. (canceled)

13. The mobile personal electronic device of claim 1, further comprising; a dedicated encryption/decryption module resident within said electronic sleeve, said encryption module resides within said sleeve CPU secured zone and is used to encrypt and decrypt said data content of said user medical records depository stored as encrypted data in said mobile device sleeve memory and the encrypted data of said two biometric sensors also being recorded and stored encrypted for further authentications as the biometric sensors registered user authentication reference data.

14. (canceled)

15. The mobile personal electronic device according to claim 3, wherein whenever any of its at least one life sign indicators detects a critical level, said mobile electronic device is configured to initiate an emergency call to at least one of a group of said mobile electronic device stored emergency mobile units wireless communication numbers and/or to remote medical service providers centers with their internet access details, and send to said memory stored emergency mobile units and/or to said remote centers a group of data files containing information associated with the user, including the identification data file of the user, personal emergency medical data file of the user, the recently measured set of pre-selected life sign parameters of the mobile electronic device user and location of the user.

16. A method for managing and updating secured Personal Health Records (PHR) containing a user's medical and health management data and associated documentation and medical imaging files such as MRI and CT, UltraSound Videos, and any other clinical and medical tests results of a single or a plurality of unique users, each one of said plurality of unique users having a mobile personal electronic device including an electronic sleeve shape add-on device uniquely associated with its said single user for storing each said single user's personal PHR files, each of said integrated personal mobile electronic devices comprising:
a. medical data combined with user's associated health management documentation and user's medical imaging files such as MRI and CT, UltraSound Videos, and any other clinical and medical tests results of a single, or a plurality of unique users wherein each one of said plurality of unique users having a mobile personal electronic device including an electronic sleeve shape add-on device uniquely associated with its said single user for storing each said single user's personal PHR files, each of said integrated personal mobile electronic devices, further comprising:
i. a mobile communication device equipped with a cellular modem to enable data communication over the cellular and internet networks equipped to receive and send user's medical data from a plurality of medical data service providers; and
ii. an electronic sleeve shaped as an add-on device, attached and fits to said mobile communication device external back side and perimeter to create together with said communication device a unified user-carried single device, wherein said electronic sleeve contains:
a biometric sensors module comprising at least two biometric/physiological sensors;
a processing module in communication with said sensor module;
an authentication unit in communication with said CPU based processing module;
an encryption module in communication with said processing module;
a memory module in communication with said processing module and said encryption module; and
communication and data connection means in communication with said mobile communication device.
the method comprising the steps of:
a. said electronic sleeve biometric sensor module reading said at least two said personal sensors output parameters of a user holding said mobile communication device;
b. said electronic sleeve biometric sensors authentication module comparing the personal biological identification parameters of said user with a pre-recorded set of identical personal biological identification parameters stored in said electronic sleeve authentication unit; and
c. if said authentication unit positively identifies said user, then allowing said user to get access to said user's PHR data documentation and medical imaging files stored in said electronic sleeve memory module and allowing during said user allowed access time duration to communicate and exchange requested user's PHR data through said communication and data connection means with external PHR data and with medical and health services providers through said mobile electronic device communication capabilities.

17. The method of claim 16 further comprising the steps of:
a. said at least two biometric sensors continuously reading said at least two personal biological identification parameters of said user while holding said mobile personal electronic device; and
b. in the event that a change occurs in any one of the measured outputs of said least two personal biological sensors identification parameters, then said invention mobile device denying access to said sleeve stored PHR data files and completely shutting down the internal stored PHR data, avoiding access and data exchange operation of said electronic sleeve content.

18. The method of claim 17, wherein at least one of said at least two biometric sensors is a life signs detector, configured to measure and record at least one of a group of life sign indicators including a heart pulse rate measurement indicator, a blood O₂ saturation level indicator, a body heat measurement indicator, an electro-dermal activity indicator, a body respiration indicator, a blood sugar level indicator, an EKG measurement indicator and a physical or emotional stress/coercion indicator, the method further comprising the steps of:
a. initiating an emergency call or an emergency communication session to at least one emergency center or at least one medical service provider that its details and communication data are registered and stored in said mobile device memory, whenever any of the life sign indicators detects through said device its user's critical abnormal level or sharp fast abnormal level change of said user's life sign;
b. wherein said emergency call transmits a selected emergency PHR data file of medical and personal information associated with the user, including identification data file of said user, personal measured recently and long term emergency medical data file of said user, including the measured set of life sign parameters of said user and the measured location of said user; and
c. whenever required said least one emergency center or said at least one medical service provider send requests for additional telemedicine tests and measurements data from said user's device, said device user gets said required requests from said center or medical service provider on his device display, then said user executes the requested measurements through said device integrated sensors and send from said device all the requested tests results back to said additional tests requesting emergency center or medical service provider which is said additional medical data request initiator.

19. The method of claim 16, wherein said integrated mobile personal electronic device further comprises an emergency button, selected from the group comprising a software generated soft button on said mobile device screen and a hardware button on said mobile device sleeve body, said button is in communication with said processing module and said communication and data connection means, the method further comprising the steps of:
a. when said emergency button is activated, communication is initiated between said mobile personal device and at least one registered emergency mobile service or emergency center at a selected medical service provider;
b. said emergency communication session by said invention mobile device transmitting a emergency data file with information associated with the user, including identification of the user, the updated personal emergency medical data file of said user, and location of said user; and
c. whenever required said least one emergency center or said at least one medical service provider send requests for additional telemedicine tests and measurements data from said user's device, said device user gets said required requests from said center or medical service provider on said device display, then said user executes the requested measurements through said device integrated sensors and send from his said device all the requested tests results back to said additional tests requesting emergency center or medical service provider, which is said additional medical data request initiator.

20. The method of claim 16, further comprising the steps of:
a. communicating via said integrated personal electronic device communication means, with any of a group of medical service providers, including at least: medical clinics, hospitals, medical insurance companies, medical imaging centers and medical testing laboratories;
b. allowing said user to remotely access and manage, change download and update said user's stored personal medical data, health management related office and billing documentation and imaging files received from data processed and stored by one or more members of said group of medical service providers; and
c. device measures periodically or upon specific data request of all remotely requested telemedicine health parameters measurements and management related parameters using said device integrated sensors and sending said tests results data files to said requesting remote medical service provider.

21. (canceled)

22. The method of claim 16, wherein said integrated mobile personal electronic device PHR content of medical data with all its associated medical documentation and medical tests results and associated imaging files, are all organized in said mobile personal device sleeve memory in several interaction layers of access permission, so said mobile personal electronic device memory content is being accessed by each medical entity under specific user's permission and after the legitimate user's is initiating the required the authentication and the identifying the required preliminary memory access enabling stage, according to the nature and legitimate needs of said medical entity treating or interacting with said user during each session, wherein said user receiving different types of medical services.

23. A PHR management system combined of a plurality of remotely distributed integrated mobile personal devices, each containing a secured personal health and medical records storage and data management module, each said integrated mobile personal device is combined of a mobile communication device operating together with an attached mobile electronic add-on sleeve device, said sleeve functioning as a private secure user bio-authentication and medical records storage and management platform, while each of said mobile communication devices communicating with at least one remote medical services provider, said system comprising:
a. a system gateway server operating as said system manager for managing and updating ID data of said system plurality of PHR data users integrated mobile personal devices and for securely communicating through the cellular networks and the internet communication infrastructure with each of said plurality of PHR data users integrated mobile personal devices and with said at least one PHR data remote medical services provider serving said PHR data users;
b. a memory sub-system connected to said system gateway server to store updated ID data of said integrated mobile personal devices and any required associated user data of each of said plurality of said integrated mobile personal devices users;
c. a plurality of integrated personal mobile devices units, each of said integrated mobile devices being associated with a unique user, each unique ID data of said mobile devices being registered with said system gateway server and wherein the ID data file of each unique ID data of said mobile devices being stored in said memory sub-system;
d. said gateway server has a plurality of registered medical emergency centers and a plurality of registered medical and health related service providers, all in secured communication through the internet network with said gateway server, said plurality of health service providers comprising at least one of the providers group including health insurance companies, clinics, hospitals, medical imaging institutes, private clinics, government health institutes and municipal health services;
e. wherein said gateway server creates access and creates a communication link with any of said selected system registered health service providers in order to get and enable the creation of a direct data access and direct communication link between said selected service provider and a selected specific user's integrated personal mobile device and exchanging and updating personal health management related PHR data and documentation files resident on said user integrated personal mobile device and wherein said access creates a two-way data transfer enabled between any of said system selected user's personal data and documentation files stored by said registered service provider and said user integrated personal mobile device PHR data storage; and
f. wherein said communication link is enabled only after positive authentication of said unique user is created by said user's integrated mobile personal device embedded user's bio-authentication capabilities.

24. The PHR management system of claim 23, wherein each of said plurality of said integrated personal mobile devices units comprises:
a. a mobile computerized communication device such as a smartphone or a notepad enabling data communication through the cellular networks and internet infrastructure and having a touch screen for user's interaction with said communication device;
b. a mobile electronic add-on sleeve device, said sleeve functioning as a private user bio-authentication and medical records secured storage and management platform physically attached and electronically interconnected with said communication device, wherein said mobile electronic sleeve device further comprising;
i. a sensor module comprising a plurality of biometric sensors for reading at least two personal biological identification parameters of the user holding the of personal identification unit, said personal identification unit being uniquely identified with said user;
ii. a processing module in communication with said sensor module for processing said personal biological identification parameters and for processing and managing the personal and secured data and documentation files associated with said user;
iii. an authentication unit in communication with the processing module configured to receive and authenticate the identity of said user by comparing said user's personal biological identification parameters read by the sensor module and processed by the processing module, with a pre-recorded set of personal biological identification parameters stored in said authentication unit;
iv. an encryption module in communication with said processing module for the encryption plus compression and/or decompression plus decryption of said user's data files to be stored in said mobile electronic sleeve device memory;
v. a memory module in communication with said processing module and said encryption module for the storage of said user's data and documentation files;
vi. communication and data connection means, in communication with said processing module for connecting said personal mobile device units with said system gateway server enabling data connection with said at least one service provider; and
vii. a set of telemedicine measurement sensors integrated into said integrated personal mobile device and data aggregated from these sensors periodically or upon request being sent to said remote service providers.

25. The PHR management system of claim 23, also further serving its multiple users as a telemedicine based platform solution, wherein each of said plurality of said integrated personal mobile devices units, further comprises;
a plurality of biological and physiological sensors integrated in each of said integrated personal mobile devices supporting the measurement and measured data processing capabilities of a plurality of user's medical and health parameters related conditions by said personal mobile device integrated sensors, said sensors measuring at least one or more of the following user's physiologic health conditions; heart bit rate measurement, blood oxygen (O₂) % content sensor measurement, EKG measurement, EEG measurement, blood sugar content measurement, body temperature measurement, blood pressure measurement, mental stress condition indication, electro-dermal activity indication, body respiration indication and body and face visible and NIR images of the user; and
said sensors measured output is transferred to selected one or more remote medical service providers by said system user's integrated personal device through said system secured communication infrastructure.
